SEQUENCE LISTING (10 sequences)

SEQ ID NO: 1 - protein, 215 aa, Artificial Sequence
  mAb CS-D7 light chain
EIVMTQSPATLSVSPGERATLSCRASQYVSDNLAWYQQKPGQAPRLLIYGASTRATGVPARFSGSGSGTEFTLTISSLQSEDFAVYYCQQYNNWRPVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 456 aa, Artificial Sequence
  mAb CS-D7 heavy chain
QVQLQESGPGLVKPSETLSLTCTVSGGSIRSSSYYWGWFRQTPGKGLEWLGNVFFSGSAYYNPSLKNRVTISIDTSENQSSLKLTSVTAADTAVYYCARPQAYSHDSSGHSPFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 11 aa, Artificial Sequence
  mAb CS-D7 VL CDR1
RASQYVSDNLA

SEQ ID NO: 4 - protein, 7 aa, Artificial Sequence
  mAb CS-D7 VL CDR2
GASTRAT

SEQ ID NO: 5 - protein, 10 aa, Artificial Sequence
  mAb CS-D7 VL CDR3
QQYNNWRPVT

SEQ ID NO: 6 - protein, 12 aa, Artificial Sequence
  mAb CS-D7 VH CDR1
GGSIRSSSYYWG

SEQ ID NO: 7 - protein, 16 aa, Artificial Sequence
  mAb CS-D7 VH CDR2
NVFFSGSAYYNPSLKN

SEQ ID NO: 8 - protein, 16 aa, Artificial Sequence
  mAb CS-D7 VH CDR3
PQAYSHDSSGHSPFDL

SEQ ID NO: 9 - protein, 123 aa, Artificial Sequence
  Humanized anti-ADDL mAb  heavy chain variable region
QVTLKESGPALVKPTQTLTLTCTLSGFSLSTSGMGVGWIRQPPGKALEWLAHIWWDDDKSYNPSLKSRLTISKDTSKNQVVLTMTNMDPVDTATYYCARRQLGLRSIDAMDYWGQGTTVTVSS

SEQ ID NO: 10 - protein, 115 aa, Artificial Sequence
  Humanized and affinity matured anti-ADDL mAb light chain variable region
DVVMTQSPLSLPVTPGEPASISCRSSQSILHSNGNTYLEWYLQKPGQSPQLLIYKVSNRFSGVPDRFSGSGSGTDFTLKISRVEAEDVGVYYCLQTTRVPLTFGQGTKLEIKRTV

(19) United States
(12) Patent Application Publication
(10) Pub. No.: US 2012/0208986 A1
(43) Pub. Date: Aug. 16, 2012

(54) USE OF MIXED MODE CHROMATOGRAPHY FOR THE CAPTURE AND PURIFICATION OF BASIC ANTIBODY PRODUCTS

(76) Inventors: Marc D. Wenger, Allentown, PA (US); Matthew Woodling, North Wales, PA (US)

(21) Appl. No.: 13/502,930

(22) PCT Filed: Oct. 13, 2010
(86) PCT No.: PCT/US10/52459

§ 371 Date: Apr. 19, 2012

Related U.S. Application Data
(60) Provisional application No. 61/253,289, filed on Oct. 20, 2009.

(51) Int. Cl. C07K 1/16; C07K 16/12

(52) U.S. Cl. 530/388.4; 530/387.1; 530/388.1

(57) ABSTRACT

Use of mixed mode chromatography for purification of an antibody from an antibody mixture, for example) a Pichia pastoris fermentation mixture containing impurities such as host cell proteins and DNA is described Mixed mode chromatography is used instead of protein A chromatography and presents certain advantages over protein A chromatography Furthermore, the integration of such a method into a multi-step procedure with other fractionation methods for purification of antibodies suitable for in vivo applications is provided.

### CROSS-REFERENCE TO RELATED APPLICATIONS

Not Applicable

### FIELD OF THE INVENTION

The present invention relates to methods for the purification of antibodies using mixed mode chromatography as a primary capture purification step. In certain embodiments, the antibody is a monoclonal antibody having an isoelectric point (pI) greater than 7.0.

### BACKGROUND OF THE INVENTION

Monoclonal antibodies are increasingly being developed and employed as therapeutics for a number of diseases, including autoimmune diseases, cancer, and infectious diseases, with over 200 antibodies in clinical trials. See Reichert, 2008, Curr Pharm Biotechnol 9:423-430.

However, monoclonal antibodies are among the most expensive of drugs with costs as high as $35,000 per year for a single patient. See Farid, 2007, J Chromatog B 848:8-18. The high cost associated with monoclonal antibodies is an impediment to its widespread usage. Therefore, reduction of costs associated with the clinical use of antibodies is critical.

One major contributor to these costs is the downstream processing, such as chromatography and filtration, necessary to purify the antibody from cell culture. See Kelley, 2007, Biotech Progress, 23: 995-1008. Downstream processing has been estimated to account for 50-80% of the total manufacturing costs of any antibody. See Roque, 2004, Biotechnol Frog 20:639-654. Due to their production in host cells, crude monoclonal antibody preparations contain many impurities, including cell components such as nucleic acids, proteins, polysaccharides, etc., as well as components of the culture media.

In a typical downstream processing scheme, a cell supernatant from cells used to produce a monoclonal antibody is clarified through the use of an initial purification step typically involving centrifugation or depth filtration. The clarified solution containing the monoclonal antibody is then separated from other proteins produced by the cell using a combination of different chromatography techniques, typically a capture chromatography step followed by polishing chromatography. See id. These techniques separate mixtures of proteins on the basis of their affinity for a biological or biomimetic ligand, charge, degree of hydrophobicity, or size. A final filtration step typically involving ultrafiltration or sterile filtration yields the final product. See id.

The capture chromatography step is commonly achieved by using affinity-based methods, which exploit a specific interaction between the antibody to be purified and an immobilized capture agent. Protein A chromatography, in particular, is typically used as the capture chromatography step in monoclonal antibody purifications. Protein A is a 41 kD cell wall protein from Staphylococcus aureas which binds with a high affinity to the Fc region of antibodies. However, the use of protein A chromatography is associated with high cost. Moreover, protein A generally cannot be cleaned with sodium hydroxide, adding to the cost associated with its use. Sodium hydroxide is a standard cleaning agent in preparative scale chromatography because it is inexpensive and effective at both cleaning and sanitizing the column for re-use. Furthermore, protein A chromatography typically requires elution at low pH, which can result in product aggregation and/or precipitation. When using protein A to purify antibodies made in yeast host cells, the protein A ligand itself is susceptible to yeast proteases, potentially resulting in higher levels of leached protein A and column deterioration.

New mixed mode chromatography ligands offer the potential for a next generation of protein purification processes by replacing a costly affinity capture step with a mixed-mode one. Mixed mode chromatography involves the use of solid phase chromatographic supports that employ multiple chemical mechanisms to adsorb proteins or other solutes. Examples of mixed mode chromatographic supports include but are not limited to chromatographic supports that exploit combinations of two or more of the following mechanisms: anion exchange, cation exchange, hydrophobic interaction, hydrophilic interaction, hydrogen bonding, pi-pi bonding, and metal affinity. Two such multi-modal ion exchange adsorbents are commercially available from GE Healthcare, the Capto Adhere™ and Capto MMC™ media. These combine strong anion and weak cation exchange groups, respectively, with hydrophobic aromatic groups.

Mixed mode chromatography supports provide unique selectivities that cannot be reproduced by single mode chromatography methods such as ion exchange. Mixed mode chromatography provides potential cost savings, longer column lifetimes and operation flexibility compared to affinity based methods. However, the development of mixed mode chromatography protocols can place a heavy burden on process development since multi-parameter screening is required to achieve their full potential. Method development is complicated, unpredictable, and may require extensive resources to achieve adequate recovery due to the complexity of the chromatographic mechanism.

What are needed are purification methods that provide for efficient purification and high yields of monoclonal antibodies.

Citation or identification of any reference in this section or any other section of this application shall not be construed as an indication that such reference is available as prior art to the present invention.

### SUMMARY OF THE INVENTION

The present invention relates to the efficient purification of antibodies, particularly monoclonal antibodies, having an isoelectric point greater than 7.0. In particular, purification conditions have been identified applicable for monoclonal antibodies having an isoelectric point of about 9.0 which provide for greatly improved purity and yields without the use of protein-A chromatography or another similar affinity chromatography step.

In one embodiment, the present invention provides a method of purifying a antibody having a pI of greater than 7.0 comprising the steps of (a) applying a mixture containing said monoclonal antibody onto a solid phase comprising a mixed mode resin having a negatively charged part and a hydrophobic part; (b) eluting said monoclonal antibody with an elution buffer containing at least about 0.3 M sodium chloride or about 0.2 to about 0.4 M sodium sulfate, buffered at pH about 7.8 to about 8.5; and (c) recovering said antibody from the mixed mode resin. In certain embodiments, eluting is performed by i) step elution with an elution buffer containing ≧about 0.3 M sodium chloride or about 0.2 to about 0.4 M sodium sulfate; or ii) gradient elution from about 0.3 to about 0.4 M sodium chloride. In certain embodiments, the eluting is performed by step elution with an elution buffer containing about 0.5 to about 1.0 M NaCl buffered at pH 8.0±0.1. In some embodiments, the eluting is performed by step elution with an elution buffer containing 0.5±0.1 M NaCl at pH 8.0±0.1. In certain embodiments, the eluting is performed by gradient elution from 0 to 1.0 M NaCl buffered at pH 8.0±0.1 over about 5 to about 20 column volumes.

In one embodiment, the antibody has a pI of about 8.5 or greater, from about 8.5 to about 10.5, or about 8.5 to about 9.5. In certain embodiments, the antibody is the S. aureus monoclonal antibody CS-D7 having a heavy chain variable region comprising the amino acid sequence as set forth in SEQ ID NO: 1 and a light chain variable region comprising the amino acid sequence as set forth in SEQ ID NO: 2. In other embodiments, the antibody is an anti-ADDL monoclonal antibody having a heavy chain variable region comprising the amino acid sequence as set forth in SEQ ID NO: 9 and a light chain variable region comprising the amino acid sequence as set forth in SEQ ID NO: 10.

Suitable buffers include, but are not limited to, HEPES, TRIS, MOPS, phosphate, BICINE, and triethanolamine.

In certain embodiments, prior to step b), the solid phase is washed with a wash buffer selected from the group consisting of Tris, HEPES, phosphate, or MOPS (the buffer having no sodium chloride), at a pH of about 7.0 to about 8.0 and a conductivity<5 mS/cm.

In certain embodiments, the method further comprises a step of purification or filtration, such as ultrafiltration or sterile filtration, after step b).

The mixed mode chromatographic support preferably has a negatively charged part that is an anionic carboxylate or sulfo group for cation exchange. In one embodiment, the solid phase comprising a mixed mode resin is Capto MMC™.

In certain embodiments, the mixture is a clarified fermentation broth from a host cell. The mixture can be subjected to a pH based precipitation. The fermentation broth can be clarified by centrifugation, depth filtration, microfiltration, pH based precipitation or any combination of the above. The clarified fermentation broth can be applied directing without adjusting the pH or conductivity or can be applied after adjusting the clarified fermentation broth to the pH and conductivity of the loading solution. In either of the foregoing, the pH will range from about 6.5 to about 7.5 and the conductivity will be <10 in S/cm. In some embodiments, the clarified fermentation broth has a pH 7.2±0.3.

In certain embodiments, the methods of the invention result in purification wherein ≧90% of the host cell protein and DNA are removed and the yield of monoclonal antibody is ≧70% or ≧80%. In certain embodiments, the monoclonal antibody is purified to a purity of ≧80%, ≧85%, or ≧88% as assessed by gel electrophoresis.

In particular embodiments, the host cell is a Pichia pastoris strain, which is optionally glycoengineered. Representative glycoengineered strains provide predominantly N-linked glycans of any of the following types: Man₅GlcNAc_{2 [}2.0], GlcNAc₂Man₃GlcNAc_{2 [}4.0], Gal₍₀₋₂₎GlcNAc₂Man₃ GlcNAc_{2 [}5.0] and NANA₍₁₋₂₎Gal₂GlcNAc₂Man₃GlcNAc_{2 [}6.0].

### BRIEF DESCRIPTION OF THE DRAWINGS

FIGS. 1A-B depict plots of binding capacity of two human monoclonal antibodies to Capto MMC™ as a function of sodium chloride (0 to 1 M) and pH (4 to 7): (A) an anti-ADDL mAb; (B) mAb CS-D7. The isoelectric point (pI) of both antibodies is about 9.0.

FIG. 2 incorporates a factorial design (2²) to prescreen parameter ranges (pH, salt concentration) for the binding of the purified mAb CS-D7 to the Capto MMC™ adsorbent in sodium chloride and ammonium sulfate salts.

FIGS. 3A-D depict 3-D plots showing the retention of the purified mAb CS-D7 and impurity sample on Capto MMC™ as a function of pH, salt type, and salt concentration: (A) sodium chloride; (B) ammonium chloride; (C) sodium sulfate; (0) ammonium sulfate.

FIGS. 4A-D depicts contour plots showing the retention of the purified mAb CS-D7 and impurity sample on Capto MMC™ as a function of pH and salt concentration: (A) purified mAb CS-D7 in sodium chloride (mg protein loaded/mL resin); (B) impurity sample in sodium chloride (fraction of maximum observed impurity binding); (C) purified mAb CS-D7 in ammonium chloride (mg protein loaded/mL resin); (0) impurity sample in ammonium chloride (fraction of maximum observed impurity binding). The impurity sample is a protein-A chromatography flowthrough, containing no or very low levels of mAb. Binding optima selected for examination in secondary experiments: α, pH 6.7±0.2 and no added salt; β, pH 7.1±0.2 and no added salt; χ, pH 5.5±0.2 with 0.25±0.05 M ammonium sulfate; and δ, pH 7.0±0.2 with 0.90 M±0.05 ammonium sulfate.

FIG. 5 provides an evaluation of optimal loading conditions from the ammonium sulfate screen using statistical software (Design-Expert 7.0 from Stat-Ease, Minneapolis, Minn.). Contour lines represent the desirability of the load conditions, with 1 the most optimal and 0 being the least optimal. Capacity and purification factor were weighted with equal importance, and a recovery down to 65% was allowed to cover a broader range, although >70% was desired. Response surfaces for capacity and purification factor are fit with a cubic polynomial model. The optimal conditions (for adsorbent capacity and robustness) chosen by visual inspection from the contour plots are designated on this graph by α, β, ω, and δ.

FIGS. 6A-B provides optimization of conditions for the elution of mAb CS-D7 from Capto MMC™ adsorbent: (A) effect of pH (elution with NaCl); (B) effect of salt concentration and salt type (pH 8). The protein was loaded at pH 7.1±0.2 and low ionic strength (no added salt). FIGS. 7A-B provides a secondary evaluation of four loading conditions in the purification of the mAb CS-D7 from the clarified supernatant by Capto MMC™ chromatography: () direct load of feed at pH 7.2 (conductivity<10 mS/cm); (V) load at pH 7.0 in 0.9 M ammonium sulfate (dialyzed); (⋄) load at pH 5.5 in 0.25 M ammonium sulfate (dialyzed); (▪) load at pH 6.75 (dialyzed, no added salt). Each column was loaded at 20 mg/mL and eluted with sodium chloride at pH 8. (A) incremental step elution with increasing sodium chloride (0.25 M/step), with the % yield of the mAb shown in the legend. (B) SDS-PAGE of each purification. The first lane is the clarified supernatant feed. Within each loading condition, the lane 1 is the nonbound fraction, and lanes 2-4 are the eluted fraction at 0 M NaCl, 0.25 M NaCl, and 0.5 M NaCl, respectively.

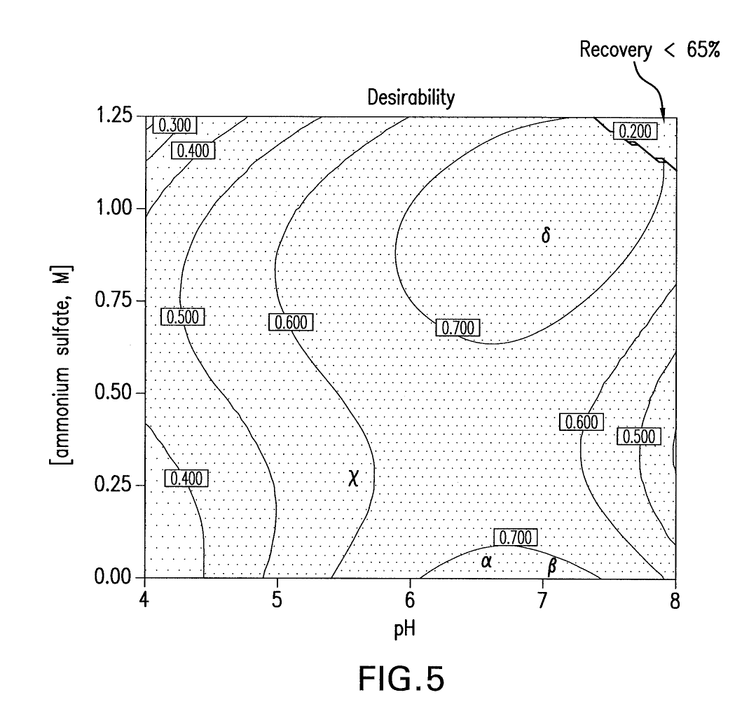

FIGS. 5A-B provides verification of the microscale chromatography (batch) results for Capto MMC™ at the laboratory column scale (1-mL Hi-trap column). (A) column chromatogram in which the clarified cell filtrate was loaded directly on the column at a residence time of four minutes. The mAb was eluted at pH 8 with NaCl in a 20-column-volume linear gradient from 0 (buffer A) to 1.0 M (buffer B). The dotted line represents host-cell protein elution as measured by ELISA. (B) SDS-PAGE (under reducing conditions) to examine the extent of purification by mixed mode chromatography: lane 1, column feed; lane 2, nonbound; lane 3, column product; lane 4, microscale chromatography (10-μL micro-tip column) product; lane 5, protein-A chromatography purified product.

### DETAILED DESCRIPTION OF THE INVENTION

The present invention relates to the use of mixed mode chromatography (e.g, Capto MMC™) as the primary capture purification step in the isolation of antibody products, particularly basic monoclonal antibodies, having an isoelectric point greater than 7.0. The antibody can be purified from any mixture containing the antibody, for example, an antibody expressed in a host cell. In an embodiment, antibody expressed from a host cell can be purified directly from the cell culture without any preceding chromatographic purification steps, such as a protein A chromatography step. Conditions were determined using the chromatography such that ≧90% of the host cell protein and DNA impurities were removed from mAb CS-D7, while maintaining a product yield of ≧75-80%.

As used herein, when used with a pH or pI (isoelectric point) value, “about” refers to a variance of 0.1, 0.2, 0.3, 0.4 or 0.5 units. When used with a temperature value, “about” refers to a variance of 1, 2, 3, 4 or 5 degrees. When used with other values, such as length and weight, “about” refers to a variance of 1%, 2%, 3%, 4% or 5%.

As used herein “antibody” refers to an immunoglobulin. The term may include but is not limited to polyclonal antibodies, monoclonal antibodies (such as those of classes IgG and IgG₂M₄), derived from human, other mammalian cell lines, or yeast such as Pichia pastoris, including natural or genetically modified forms such as humanized, human, single-chain, chimeric, synthetic, recombinant, hybrid, mutated, grafted, and in vitro generated antibodies.

As used herein, “capture chromatography” refers to the first or primary chromatography of the purification sequence, in which a column is loaded with an antibody-containing mixture, e.g., a clarified cell supernatant, and operated in an on-off mode with step or gradient elution.

As used herein, “clarified” refers to a sample (i.e. a cell suspension) having undergone a solid-liquid separation step involving one or more of centrifugation, microfiltration and depth filtration to remove host cells and/or cellular debris. A clarified fermentation broth may be a cell culture supernatant. Clarification is sometimes referred to as a primary or initial recovery step and typically occurs prior to any chromatography or a similar step.

As used herein, a “mixture” comprises an antibody of interest (for which purification is desired) and one or more contaminant, i.e., impurities. The mixture can be obtained directly from a host cell or organism producing the polypeptide. Without intending to be limiting, examples of mixtures that can be purified according to a method of the present invention include harvested cell culture fluid, cell culture supernatant and conditioned cell culture supernatant. A mixture that has been “partially purified” has already been subjected to a chromatography step, e.g., non-affinity chromatography, affinity chromatography, etc. A “conditioned mixture” is a mixture, e.g., a cell culture supernatant that has been prepared for a chromatography step used in a method of the invention by subjecting the mixture to one or more of buffer exchange, dilution, salt addition, pH titration or filtration in order to set the pH and/or conductivity range and/or buffer matrix to achieve a desired chromatography performance. A “conditioned mixture” can be used to standardize loading conditions onto the first chromatography column. In general, a mixture can be obtained through various separation means well known in the art, e.g., by physically separating dead and viable cells from other components in the broth at the end of a bioreactor run using filtration or centrifugation, or by concentration and/or diafiltration of the cell culture supernatant into specific ranges of pH, conductivity and buffer species concentration.

As used herein, “monoclonal antibody” is used in the conventional sense to refer to an antibody obtained from a population of substantially homogeneous antibodies such that the individual antibodies comprising the population are identical except for possible naturally occurring mutations that may be present in minor amounts. Monoclonal antibodies are highly specific, being directed against a single antigenic site. The term “monoclonal”, in describing antibodies, indicates the character of the antibody as being obtained from a substantially homogeneous population of antibodies, and is not to be construed as requiring production of the antibody by any particular method.

As used herein, the “pI” or “isoelectric point” of an antibody refers to the pH at which the antibody's positive charge balances its negative charge. The pI can be determined according to various conventional methodologies, e.g., calculated from the primary sequence using the net charge of the amino acid and/or sialic acid residues on the antibody or measured experimentally by using isoelectric focusing.

As used herein, “polishing chromatography” refers to one or more additional chromatographic steps following capture chromatography and is used to remove residual host cell impurities, product-related impurities (product fragments and/or aggregated species), and virus contaminants.

As used herein, a “variable region” has the structure of an antibody variable region from a heavy or light chain. Antibody heavy and light chain variable regions contain three complementarity determining regions interspaced onto a framework. The complementarity determining regions are primarily responsible for recognizing a particular epitope.

When referring to “mole percent” of a glycan present in a preparation of a glycoprotein, the term means the molar percent of a particular glycan or glycans present in the pool of N-linked oligosaccharides released when the protein preparation is treated with PNG'ase and then quantified by a method that is not affected by glycoform composition, (for instance, labeling a PNG'ase released glycan pool with a fluorescent tag such as 2-aminobenzamide and then separating by high performance liquid chromatography or capillary electrophoresis and then quantifying glycans by fluorescence intensity). For example, 50 mole percent NANA₂Gal₂GlcNAc₂Man₃GlcNAc₂ means that 50 percent of the released glycans are NANA₂Gal₂GlcNAc₂Man₃GlcNAc₂ and the remaining 50 percent are comprised of other N-linked oligosaccharides. As another example, 50 mole percent NANA₁₋₂Gal₂GlcNAc₂Man₃GlcNAc₂ means that 50 percent of the released glycans are a mixture of NANA₁Gal₂GlcNAc₂Man₃GlcNAc₂ and NANA₂Gal₂GlcNAc₂Man₃GlcNAc₂ and the remaining 50 percent are comprised of other N-linked oligosaccharides. In various embodiments, the mole percent of a particular glycan or glycans in a preparation of glycoprotein will be between 20% and 100%, preferably above 25%, 30%, 35%, 40% or 45%, more preferably above 50%, 55%, 60%, 65% or 70% and most preferably above 75%, 80% 85%, 90% or 95%.

As used herein, the term “predominantly” or variations such as “the predominant” or “which is predominant” will be understood to mean the glycan species, or a combination of glycan species, that has the highest mole percent (%) of total N-glycans after the glycoprotein has been treated with PNGase and released glycans analyzed by mass spectroscopy, for example, MALDI-TOF MS. In other words, the phrase “predominantly” can be defined as an individual entity, such as a specific glycoform, or can be defined as a combination of specific glycoforms, that is present in greater mole percent than any other entity or entities. For example, if a composition consists of species A in 40 mole percent, species B in 35 mole percent and species C in 25 mole percent, the composition comprises predominantly species A.

Antibodies

The present invention provides methods for purification of antibodies, particularly monoclonal antibodies, having an isoelectric point greater than 7.0. In certain embodiments, the monoclonal antibody has an isoelectric point of about 8.5 or greater, about 8.5 to about 10.5, about 8.5 to 10.0, or about 8.5 to 9.5. In certain embodiments, the monoclonal antibody has an isoelectric point of about 9.0 to about 11.0 or of about 9.0 to about 10.0. In other embodiments, the monoclonal antibody has an isoelectric point of 9.0±0.5, 9.0±0.4, 9.0±0.3, or 9.0±0.2.

In certain embodiments, the monoclonal antibody is a S. aureus CS-D7 target region specific monoclonal antibody. The CS-D7 target region provides an S. aureus ORF0657n (also known as IsdB) epitope that can be targeted to reduce the likelihood or severity of an S. aureus infection. See PCT International Publication No. WO 2009/029132. The CS-D7 target region is specifically targeted by monoclonal antibody CS-D7 (mAb CS-D7). MAb CS-D7 is an immunoglobulin having two light chains with an amino acid sequence of SEQ ID NO: 1 and two heavy chains with an amino acid sequence of SEQ ID NO: 2.

The variable regions for mAb CS-D7 and corresponding CDR SEQ ID NOs are summarized in Table 1.

TABLE 1

| | Light Chain Variable Region | | | Heavy Chain Variable Region | | |
|---|---|---|---|---|---|---|
| | CDR₁ | CDR₂ | CDR₃ | CDR₁ | CDR₂ | CDR₃ |
| | SEQ ID | SEQ ID | SEQ ID | SEQ ID | SEQ ID | SEQ ID |
| mAb | NO: | NO: | NO: | NO: | NO: | NO: |
| CS-D7 | 3 | 4 | 5 | 6 | 7 | 8 |

Basic antibodies (pI≧7), in particular those having an isoelectric point of about 8.5 or about 9.0 or greater, are expected to behave similarly to mAb CS-D7. Monoclonal antibodies targeting S. aureus CS-D7 that can be purified using the methods of the invention have hydrophobic and electrostatic properties similar to mAb CS-D7. A small number of amino acid changes, as contemplated herein, is expected to have no effect on mixed mode chromatography. Glycosylation patterns that do not impact the pI of the molecule substantially (i.e., <0.2 units) are expected to have little or no effect on mixed mode chromatography. Increasing degrees of sialic acid occupancy could substantially change the pI of the molecule. Therefore, care should be taken when modifying sialic acid occupancy.

Antibodies targeting the CS-D7 region include those described in PCT International Application Publication No. WO 2009/029132, including mAbs designated CS-E11, CS-D10, CS-A10, BMV-H11, BMV-E6, BMV-D4, and BMV-C2.

Additional monoclonal antibodies targeting the CS-D7 target region can be obtained using full-length ORF0657n or a polypeptide that provides the epitope recognized by mAb CS-D7 (located within approximately amino acids 42-285 of ORF0657n). A variety of techniques are available to select for a protein recognizing an antigen. Examples of such techniques include the use of phage display technology and hybridoma production. In the hybridoma method, a mouse or other appropriate host animal, such as a hamster or macaque monkey, is immunized with an antigen to elicit lymphocytes that produce or are capable of producing antibodies that will specifically bind to the antigen used for immunization. Suitable hybridoma techniques are known in the art. See, e.g., Harlow et al., Antibodies, A Laboratory Manual, Cold Spring Harbor Laboratory, 1988.

In certain embodiments, the monoclonal antibody is affinity matured humanized anti-ADDL monoclonal antibody, See U.S. Patent Application Publication No. US20060228349. The affinity matured humanized mAb is an immunoglobulin having two light chains with an amino acid sequence of SEQ ID NO: 9 and two heavy chains with an amino acid sequence of SEQ ID NO: 10.

Amino acid changes, which can be any combination of amino acid deletions, insertions or substitutions, may be incorporated into any of the antibodies described above. The substituted amino acids should have one or more similar properties such as charge, size, polarity and/or hydrophobicity. Amino acid changes can be selected to alter pKi, e.g., to make an antibody more basic, preferably at amino acids which are solvent exposed. The number of amino acid changes in the light chain or heavy chain sequence may be 1, 2, 3, 4, 5, 6, 7, 8, 9 or 10 amino acids. Conservative substitutions are preferred.

Host Cells

The antibody of interest can be produced or expressed by living host cells that have been genetically engineered to produce the protein. Methods of genetically engineering cells to produce proteins are well known in the art. See e.g. Ausabel et al., eds. (1990), Current Protocols in Molecular Biology (Wiley, New York) and U.S. Pat. Nos. 5,534,615 and 4,816,567. Such methods include introducing nucleic acids that encode and allow expression of the protein into living host cells. These host cells can be bacterial cells, fungal cells, or, preferably, animal cells grown in culture. Bacterial host cells include, but are not limited to E. coli cells. Examples of suitable E. coli strains include: HB1O1, DH5α, GM2929, JM109, KW251, NM538, NM539. Fungal host cells that can be used include, but are not limited to, Saccharomyces cerevisiae, Pichia pastoris and Aspergillus cells. A few examples of animal cell lines that can be used are CHO, VERO, DXB11, BHK, HeLa, Cos, MDCK, 293, 3T3, NSO and WI138. New animal cell lines can be established using methods well know by those skilled in the art (e.g., by transformation, viral infection, and/or selection). In particular embodiments, the protein of interest is produced in a CHO cell. Various types of CHO cells are known in the art, e.g., CHO-K1, CHO-DG44, CHO-DXB11, CHO/dhfr⁻ and CHO-S. A host cell that has been engineered with nucleic acid encoding the protein of interest can be cultured under conditions well known in the art that allow expression of the protein.

Lower eukaryotes, particularly yeast and filamentous fungi, can be genetically modified so that they express glycoproteins in which the glycosylation pattern is human-like or humanized. In this manner, glycoprotein compositions can be produced in which a specific desired glycoform or combination of glycoforms is predominant in the composition. Such can be achieved by eliminating selected endogenous glycosylation enzymes and/or genetically engineering the host cells and/or supplying exogenous enzymes to mimic all or part of the mammalian glycosylation pathway as described in U.S. Pat. Nos. 5,714,377; 7,029,872; 7,198,921 and 7,259,007, U.S. Patent Application Publication Nos. 2004/0018590, 2004/074458, 2005/0170452, 2005/0260729, 2006/0040353, 2006/0211085, 2006/0286637, and 2007/0037248, and PCT International Application Publication No. WO 2007/061631.

In certain embodiments, Pichia pastoris host cells include those that have been genetically modified to produce glycoproteins that have no N-glycans compositions wherein the predominant N-glycan is selected from the group consisting of complex N-glycans, hybrid N-glycans, and high mannose N-glycans wherein complex N-glycans are selected from the group consisting of Man₃GlcNAc₂, GlcNAC₍₁₋₄₎Man₃GlcNAc₂, Gal₍₀₋₄₎GlcNAc₍₁₋₄₎Man₃GlcNAc₂, and NANA₍₁₋₄₎Gal₍₁₋₄₎GlcNAc₍₀₋₂₎Man₃GlcNAc₂; hybrid N-glycans are selected from the group consisting of Man₅GlcNAc₂, GlcNAcMan₅GlcNAc₂, GalGlcNAcMan₅GlcNAc₂, and NANAGalGlcNAcMan₅GlcNAc₂; and high Mannose N-glycans are selected from the group consisting of Man₆GlcNAc₂, Man₇GlcNAc₂, Man₈GlcNAc₂, and Man₉GlcNAc₂.

### Mixed Mode Chromatography Supports

Mixed mode chromatography refers to chromatography that substantially involves a combination of two or more chemical mechanisms. In some embodiments, the combination results in unique selectivities such that it is able to achieve fractionation among antibodies that cannot be achieved by a single mode support. In certain embodiments, the mixed-mode resin comprises a negatively charged part and a hydrophobic part. In one embodiment, the negatively charged part is an anionic carboxylate group or anionic sulfo group for cation exchange. Examples of such supports include, but are not limited to, Capto-MMC™ (GE Healthcare). See Table 1.

Various other mixed mode chromatography media are available commercially. While mixed-mode resins that do not comprise a negatively charged part and a hydrophobic part are not expected to behave similarly to Capto-MMC™, optimal conditions can be determined for other mixed mode resins using the methods described herein. Commercially available examples include but are not limited to ceramic hydroxyapatite (CHT) or ceramic fluorapatite (CFT), MEP-Hypercel™, Capto-Adhere™, Bakerbond™ Carboxy-Sulfon™ and Bakerbond™ ABx™ (J. T. Baker). See Table 1.

TABLE 1
Summary of Mixed Mode Chromatography Media

| | Proposed | | |
|---|---|---|---|
| Adsorbent Name | Mechanism | Base Matrix | Ligand Chemistry |
| GE Healthcare Capto MMC™ | Weak cation- exchange and hydrophobic interaction | Agarose | 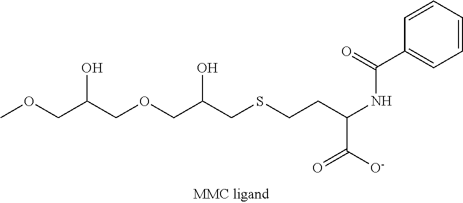 |
| GE Healthcare Capto Adhere | Strong anion- exchange and hydrophobic interaction | Agarose | 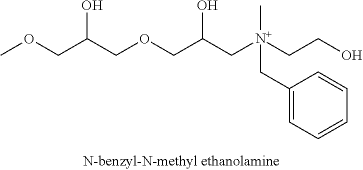 |
| Pall MEP Hypercel | Hydrophobic charge induction (pKa = 4.8, antibody selective ligand) | Cellulose | 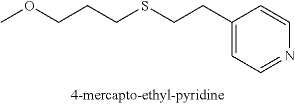 |
| Pall HEA HyperCel | Hydrophobic charge induction (pKa = 8.0, aliphatic substituent) | Cellulose | 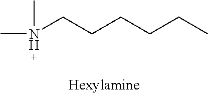 |
| Pall PPA HyperCel | Hydrophobic charge induction (pKa = 8.0, aromatic substituent) | Cellulose | 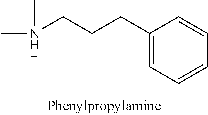 |
| Baker Bond | Strong and weak | Silica | —COOH and —S groups |
| ABx | cation exchange | | |
| Baker Bond | Weak cation and | Polymeric | —COOH and polyethylene imine (—C₂H₅N) |
| ABx | weak anion exchange | resin | |
| Bio-Rad | Cation-exchange and | Hydroxyapatite | Ca₁₀(PO₄)₆(OH)₂ |
| Ceramic | coordination bonds | | |
| Hydroxyapatite | (between Ca²⁺ and | | |
| | carboxy/phosphoryl | | |
| | groups) | | |

The chromatograph support may be practiced in a packed bed column, a fluidized/expanded bed column, and/or a batch operation where the mixed mode support is mixed with the antibody preparation for a certain time. A solid phase chromatography support can be a porous particle, nonporous particle, membrane, or monolith. The term “solid phase” is used to mean any non-aqueous matrix to which one or more ligands can adhere or alternatively, in the case of size exclusion chromatography, it can refer to the gel structure of a resin. The solid phase can be any matrix capable of adhering ligands in this manner, e.g., a purification column, a discontinuous phase of discrete particles, a membrane, filter, gel, etc. Examples of materials that can be used to form the solid phase include polysaccharides (such as agarose and cellulose) and other mechanically stable matrices such as silica (e.g. controlled pore glass), poly(styrenedivinyl)benzene, polyacrylamide, ceramic particles and derivatives of any of these.

In some embodiments, the mixed mode support is packed in a column of at least 5 mm internal diameter and a height of at least 25 mm. Such embodiments are useful, e.g., for evaluating the effects of various conditions on a particular antibody.

Another embodiment employs the mixed mode support, packed in a column of any dimension required to support preparative applications. Column diameter may range from less than 1 cm to more than 1 meter, and column height may range from less than 1 cm to more than 30 cm depending on the requirements of a particular application. Commercial scale applications will typically have a column diameter (ID) of 20 cm or more and a height of at least 25 cm.

Appropriate column dimensions can be determined by the skilled artisan.

### Description of the Method

The methods of the invention can be used for any mixture containing an antibody of interest. For example, antibody preparations to which the invention can be applied can include unpurified or partially purified antibodies from natural, synthetic, or recombinant sources. A mixture comprising a desired antibody may be any composition or preparation, such as, for example, a body fluid derived from a human or animal, or a fluid derived from a cell culture, such as, for example, a cell culture supernatant or cell culture harvest. The mixture may be cell culture material, for example, solubilized cells, such as cell culture supernatant. It certain embodiments, it is a clarified cell culture harvest. Clarification typically involves centrifugation to pellet the cell solids and recover the supernatant for further processing. Alternatively, microfiltration, depth filtration, or a pH based precipitation (acid or base) may be used to filter away the cell solids, with the filtrate recovered for further purification. Clarification can also involve a combination of these steps, for example, centrifugation coupled with microfiltration or depth filtration. See, e.g., Wang et al., 2006, Biotechnol Bioeng 94:91-104. Furthermore, it may also be a fluid derived from another purification step such as ultrafiltration for impurity clearance and/or buffer exchange.

The first step of the process herein involves applying an antibody mixture such as a yeast (e.g., Pichia pastoris) fermentation broth or mammalian cell culture supernatant, which may be clarified, containing the antibody onto a solid phase comprising a mixed mode resin containing ligands which comprise a negatively charged part and a hydrophobic part. In some embodiments, the mixed mode resin is in a column. The column may be low-pressure (≦3 bar of back pressure). In some embodiments, the column is packed with a medium having a particle diameter of about >30 μm, for example, about 30 to about 100 μm. In some embodiments, the column has a pore size of about 100 to about 4000 angstroms, for example, about 150 to about 300 angstroms. In some embodiments, the column length is about 10 to about 50 cm, for example, about 25 to about 35 cm. Preferably, the column is a preparative column, meaning preparative scale and/or preparative load. The preparative-scale column typically has a diameter of at least about 1 cm, for example, at least about 6 cm, up to and including about 15 cm, about 60 cm, or higher. The medium of the column may be any suitable material, including polymeric-based media, silica-based media, or methacrylate media. In one embodiment, the medium is agarose-based.

The column may be an analytical or preparative column. The amount of antibody loaded onto the column is generally about 0.01 to about 40 g antibody/liter bed volume, for example, about 0.02 to about 30 g antibody/liter bed volume, about 1 to about 25 g molecule/liter bed volume, or about 3 to about 25 g antibody/liter bed volume. The preparative-load column has a load of molecule of at least about 0.1 g antibody/liter bed volume, for example, at least about 1 g antibody/liter.

The flow rate is generally about 50 to about 600 cm/hour, or about 4 to about 20 column volumes (CV)/hour, depending on the column geometry. Appropriate flow velocity can be determined by the skilled artisan.

The temperature may be in the range of about 4 to about 30° C., such as room temperature.

In preparation for contacting the antibody preparation with the mixed mode support, in some embodiments, the chemical environment inside the column is equilibrated. This is commonly accomplished by flowing an equilibration buffer that is equivalent or similar to the loading buffer conditions, such as a MES, MOPS, HEPES, potassium phosphate or sodium phosphate buffer solution with or without sodium chloride, through the column to establish the appropriate pH; conductivity; and other pertinent variables. The equilibration buffer is preferably isotonic with a conductivity≦10 mS/cm and commonly has a pH in the range from about 6 to about 8.

In preferred embodiments, the antibody mixture is directly applied to the column without any adjustment of pH or conductivity. However, in certain embodiments, the mixture comprising the antibody can be adjusted to a pH of about 6 to about 8, for example, about 7 to about 7.5, and/or as an alternative diluted with water or weak buffer to a conductivity of less than about 10 mS/cm at about pH 7. This is essential to allow binding of the antibody to the cation-exchange resin. Adjustment of pH can be performed by addition of buffer such as Tris, phosphate, HEPES, MOPS, or MES. The exact concentration and pH of this added buffer will depend on the pH and buffer matrix of the fluid (cell filtrate) being adjusted. Adjustment of conductivity can be performed by dilution with water or weak buffer such as 10 mM phosphate. Preferably, in either of the foregoing embodiments, the cell filtrate has a pH of 7.2±0.2 and a conductivity<10 mS/cm.

An elution buffer is used to elute the antibody from the mixed mode resin. Suitable elution buffers include but are not limited to HEPES, TRIS, phosphate, BICINE, or triethanolamine containing at least 0.3 M sodium chloride or about 0.2 to about 0.4 M sodium sulfate, buffered at pH 7.8 to 8.5. In certain embodiments, the elution buffer contains at least 0.4 M sodium chloride. The antibody may be eluted with step elution with an elution buffer having a salt concentration higher than the salt concentration of elution or by gradient elution with any gradient starting with a salt concentration below the salt concentration of elution. For the monoclonal antibodies CS-D7 and anti-ADDL mAB, the salt concentration of elution is about 0.3 to about 0.4 M. In specific embodiments, elution occurs by i) step elution with an elution buffer containing ≧0.3 M sodium chloride or about 0.2 to about 0.4 M sodium sulfate; or ii) gradient elution from about 0.3 to about 0.4 M sodium chloride. The elution buffer for step elution may contain about 0.5 to about 1.0 M NaCl, for example, 0.5±0.1 M NaCl, buffered at pH 8.0±0.1. An elution buffer for gradient elution may be any gradient that encompasses, for example, about 0.3 to about 0.4 M, including, but not limited to, 0 to about 1.0 M, 0 to about 0.5 M, about 0.1 to about 0.5 M, or about 0.1 to about 0.4 M NaCl. The elution buffer is typically buffered at pH 8.0±0.1. Elution typically occurs over about 5 to about 20 column volumes.

After use, the mixed mode column may optionally be cleaned, sanitized, and stored in an appropriate agent, and optionally, re-used.

In certain embodiments, the chromatographic support is optionally washed after loading. The column can be washed to 1) remove unbound loading sample from the column prior to elution and 2) remove weakly bound impurities. For example, if loading occurs at pH 7, a wash at pH 8 without NaCl, may wash off some bound impurities prior to increasing salt strength for product elution. Wash strategies are often used in lieu of a gradient elution at process scale since they are simple to implement. The wash buffer typically contains Tris, HEPES, phosphate, BICINE, or triethanolamine, having a pH between about 7.5 and about 8.0 and a conductivity<5-10 mS/cm.

The mixed mode chromatography is preferably used as a capture step, and thus serves for purification of a monoclonal antibody, in particular to the reduction, decrease or elimination, of host cell proteins, antibody aggregates and antibody fragments and for concentration of a monoclonal antibody preparation.

Purified when referring to a component or fraction indicates that its relative concentration (weight of component or fraction divided by the weight of all components or fractions in the mixture) is increased by at least about 20%. In one series of embodiments, the relative concentration is increased by at least about 40%, about 50%, about 60%, about 75%, about 100%, about 150%, or about 200%. A component or fraction can also be said to be purified when the relative concentration of components from which it is purified (weight of component or fraction from which it is purified divided by the weight of all components or fractions in the mixture) is decreased by at least about 20%, about 40%, about 50%, about 60%, about 75%, about 85%, about 95%, about 98% or 100%. In still another series of embodiments, the component or fraction is purified to a relative concentration of at least about 50%, about 65%, about 75%, about 85%, about 90%, about 97%, about 98%, or about 99%.

In preferred embodiments, using the methods of the invention, ≧90% of the host cell protein and DNA is removed and the yield of monoclonal antibody is ≧70%, more preferably ≧80%.

In preferred embodiments, a monoclonal antibody is purified to a purity of ≧80% or ≧85% as assessed by gel SDS electrophoresis. Electrophoresis is generally carried out under denaturing and reducing conditions, using a polyacrylamide gel such as a 4-12%, 4-20%, 10%, or 12% gel in a NuPAGE or Tris-glycine gel system such as from Invitrogen (Carlsbad, Calif.). Gels are stained overnight with a suitable stain, for example, Sypro Ruby fluorescent protein stain from Invitrogen and then imaged with a laser-induced fluorescence scanner such as a Molecular Dynamics fluoroimager 595.

### Additional Optional Steps

The eluted protein preparation may be subjected to additional purification or filtration steps either prior to, or after, the mixed mode chromatography step. Exemplary further purification steps include hydroxyapatite chromatography; dialysis; membrane ultrafiltration, affinity chromatography using an antibody to capture the protein; hydrophobic interaction chromatography (HIC); mixed-mode chromatography; ammonium sulphate precipitation; anion or cation exchange chromatography; ethanol precipitation; reverse phase HPLC; chromatography on silica; chromatofocusing; and gel filtration.

In certain embodiments, a further purification and filtration step occurs after eluting from the mixed mode resin. Such purification preferably involves one or more polishing chromatography steps (such as cation or anion exchange chromatography, hydrophobic interaction chromatography, or hydroxyapatite chromatography) to remove residual host cell proteins, nucleic acids, and viruses as well as process excipients such as protease inhibitors and leached mixed-mode ligand. The filtration step preferably involves ultrafiltration for removal of low molecular-weight impurities and process excipients and for exchange into the final product formulation buffer. A sterile filtration occurs to remove any particulates and to control bioburden (as required).

The specific embodiments described herein are offered by way of example only, and the invention is to be limited only by the terms of the appended claims along with the full scope of equivalents to which such claims are entitled. Indeed various modifications of the invention, in addition to those shown and described herein will become apparent to those skilled in the art from the foregoing description and accompanying drawings. Such modifications are intended to fall within the scope of the appended claims.

### EXAMPLES

### Purification Reagents

Sodium acetate, sodium phosphate, sodium chloride, ammonium chloride, sodium sulfate, sodium ammonium sulfate, and methanol were supplied by Fisher Scientific (Pittsburgh, Pa.). HEPES buffer was purchased from Sigma-Aldrich (St. Louis, Mo.). A universal buffer mixture of 10 mM sodium acetate, 10 mM sodium phosphate, and 10 mM HEPES buffer was used in the screening experiments to evaluate a broad pH range between 4 and 8, with the desired pH achieved by titration with sodium hydroxide or hydrochloric acid. Capto MMC™ chromatography media was obtained from GE Healthcare (Upsala, Sweden).

### Micro-Tip Columns

Micro-tip columns (PhyTips®) containing 10 μL of immobilized Capto MMC™ adsorbent at the bottom of 1-mL clear-plastic pipette tips were supplied by PhyNexus (San Jose, Calif.). The columns were prepared by a procedure in which a pre-defined volume of adsorbent slurry is drawn up for a given column volume and then situated between two hydrophilic frits which are welded to the bottom of the pipette tip. Following their manufacture, the micro-tip columns were then stored in 50% glycerol. These columns have a tapered shape, with an inner diameter (i.d.) at top of the column of approximately 2 mm, an i.d. at the bottom of approximately 1 mm, and a bed height of 5.5 mm.

### Labware

The micro-tip chromatography experiments were performed in BD Falcon 96-deepwell microplates (1.0-mL/well and 2.0 mL/well) with square pyramid bottoms purchased from Fisher Scientific (Pittsburgh, Pa.). For samples requiring temperature control, the chromatography was performed in 2-mL Nalgene cyrovials (Fisher Scientific) on the temperature-controlled rack. Micro-tip pre-washing steps were carried out in 12-column partitioned reservoir plates from Seahorse Bioscience (Chicopee, Mass.). Eight-row or twelve-column partitioned reservoir plates (Seahorse Bioscience) and 100-mL troughs (Tecan US, Inc., Durham, N.C.) were used for holding buffered solutions required for the microplate chromatography.

Samples were placed on the Tecan Freedom Evo liquid-handling robot either in 2-mL Nalgene cryovials or 15-mL Falcon polypropylene centrifuge tubes (Fisher Scientific, Pittsburgh, Pa.). Black liquid-sensing (non-sterile) disposable pipette tips (200 and 1000 μL) were purchased from Axygen (Union City, Calif.). UV-transparent BD Falcon (300 μL/well) or Corning Costar (150 μL/well) 96-well microplates were purchased from Fisher Scientific for making absorbance readings at λ=280 nm.

### Analysis

Purified antibody stock and product pool concentrations were determined by absorbance (280 nm) using an Agilent spectrophotometer (Santa Clara, Calif.). Real-time absorbance (280 nm) measurements of fractions from the microscale experiments were made in a UV-transparent 96-halfwell microplate in the Tecan Ultra384 plate reader. Total protein concentration of experimental samples was determined by the Bicinchoninic Acid (BCA) assay kit from Pierce (Rockford, Ill.) in a 96-well format carried out on a Tecan Genesis 150 workstation. Residual DNA concentration was quantified in experimental samples using the Quant-iT™ PicoGreen® dsDNA Assay Kit from Invitrogen (Carlsbad, Calif.), also automated on a Tecan Genesis 150 workstation. Chromatographic samples were also analyzed by SDS-PAGE for purity. Electrophoresis was carried out under denaturing and reducing conditions, using a 4-12% gradient NuPAGE gel system from Invitrogen (Carlsbad, Calif.). Gels were stained overnight with Sypro Ruby fluorescent protein stain (Invitrogen).

Antibody concentration in crude and purified samples was analyzed by bio-layer interferometry using the ForteBio (Menlo Park, Calif.) Octet QK system with protein A biosensors. A 16-point calibration curve ranging from 1 to 300 μg/mL was prepared in 50 mM HEPES (pH 7.5), 150 mM NaCl, 0.05% polysorbate-20, and 1% BSA and fit with a 4-parameter logistic equation. Samples were diluted 5 to 50-fold in the HEPES assay diluent, and the unknown sample concentrations were determined by interpolation from the curve.

Host cell proteins (HCP) from Pichia pastoris were quantified by ELISA on a Tecan Evo200 workstation using anti-HCP polyclonal antibodies (pAbs) from Cygnus Technologies (Southport, N.C.). Briefly, microplates were coated with 5 μg/mL of the anti-host pAbs, followed by the addition of reference standards and samples. Following the capture step, biotinylated anti-HCP pAbs were added to the plate, forming an immune complex. This complex was detected by the addition of streptavidin-alkaline phosphatase (AP) conjugate and the fluorogenic substrate, 4-methylumbelliferyl phosphate (4-MUP). A standard curve is generated by plotting fluorescence intensity vs. concentration. The curve is fit with a four-parameter logistic equation, and unknown sample concentrations was determined by interpolation from the curve.

### Microscale Chromatography

Microscale chromatography was carried out in batch mode by pipetting a sample or solution back and forth across a micro-tip column. Sample and purification reagents (pre-wash, equilibration, wash, and elution buffers) were presented in a 96-well microplate, and the micro-tip columns were then moved across the plate during the purification. For the pre-wash, equilibration, and wash steps, a single aspiration-dispense cycle per solution was performed, although multiple aliquots of wash or equilibration buffer were usually used. For the load and elution steps, multiple aspiration-dispense cycles were carried out with each aliquot to increase the contact time, with load times of ≧20 minutes and elution times of ≧5 minutes per well. All equilibration, loading, and wash solutions were buffered with a mixture of 10 mM sodium acetate, 10 mM sodium phosphate, and 10 mM HEPES (enabling a useful pH range from 4 to 8). The elution solutions were buffered only with HEPES. The free acid and sodium salts of the respective buffer components were blended accordingly to achieve the desired pH of the specific condition being examined. Buffered solutions also containing either sodium chloride, ammonium chloride, sodium sulfate, or ammonium sulfate were prepared to examine these salts across a pH range.

Liquid-handling parameters (liquid classes) for micro-tip operation were developed on the Tecan for accurate and reproducible flow and to operate in a flow regime that matches the typical linear velocities of laboratory-scale chromatography. Specifically, this involved decreasing the pipetting flow rates to between 2 and 20 μL/sec, considerably lower than those normally used for pipetting fluids (150-600 μL/sec). For the aspiration and dispense positions, the micro-tip column was set to pipette 1 mm (10 steps) above the bottom of the microwell (z-max). To minimize contamination of droplets of system liquid (water) falling into the micro-tip columns, an airgap of 30 μL was positioned between the system liquid and the bottom of the DiTi adapter cone. However, this air gap can break down after multiple aspiration-dispense cycles and therefore requires a periodic water flush to re-establish the air gap.

Prior to use, the Capto MMC™ micro-tip columns were pre-washed in 12-column trough plates the following sequence: 0.5 mL/micro-tip of 50% methanol, 0.8 mL/micro-tip of water, 0.2 mL/micro-tip of elution buffer; and 0.8 mL/micro-tip of water. The micro-tips columns were then equilibrated in 96-well deepwell plates with 4 aliquots×0.85 mL of the respective loading buffer. Purification buffers were pre-dispensed before each chromatography experiment from 100-mL troughs, or from 8-row partitioned-reservoir plates when the buffer varied with each micro-tip column. The specific volume of each aliquot is an operating parameter that is optimized for a particular purification. To avoid introducing air into the micro-tip bed, the aspiration volume through the micro-tip column was 50 μL less than aliquot volume to account for the well hold-up volume.

### Example 1

### Complexity of Design Space in Mixed Mode Chromatography

Mixed mode chromatographic adsorbents offer the promise of increased selectivity and more salt-tolerant loading conditions in the purification of biomolecules including monoclonal antibodies. However, their design space is considerably more complex and the conditions for optimal operation less predictable.

In order to get a better understanding of the differences, two different human monoclonal antibodies (mAb), each of the IgG1-κ subclass with a pI of about 9.0, were used with Capto MMC™. The first monoclonal antibody (an anti-ADDL mAb) targets Amyloid Beta-Derived Diffusible Ligand. See U.S. Patent Application Publication No. 2006/0228349. The anti-ADDLs mAb was expressed in CHO bioreactors and purified from clarified supernatant by standard chromatography (protein A and ion-exchange chromatography) and filtration processes. See Kelley, 2007, Biotech Progress 23:995-1008; Shukla et al., 2007, J Chromatogr B 848:28-39. The second mAb is the CS-D7 mAb, which was expressed in CHO cells and alternatively in Pichia Pastoris and then purified from clarified fermentation supernatant by standard chromatography methods as indicated above.

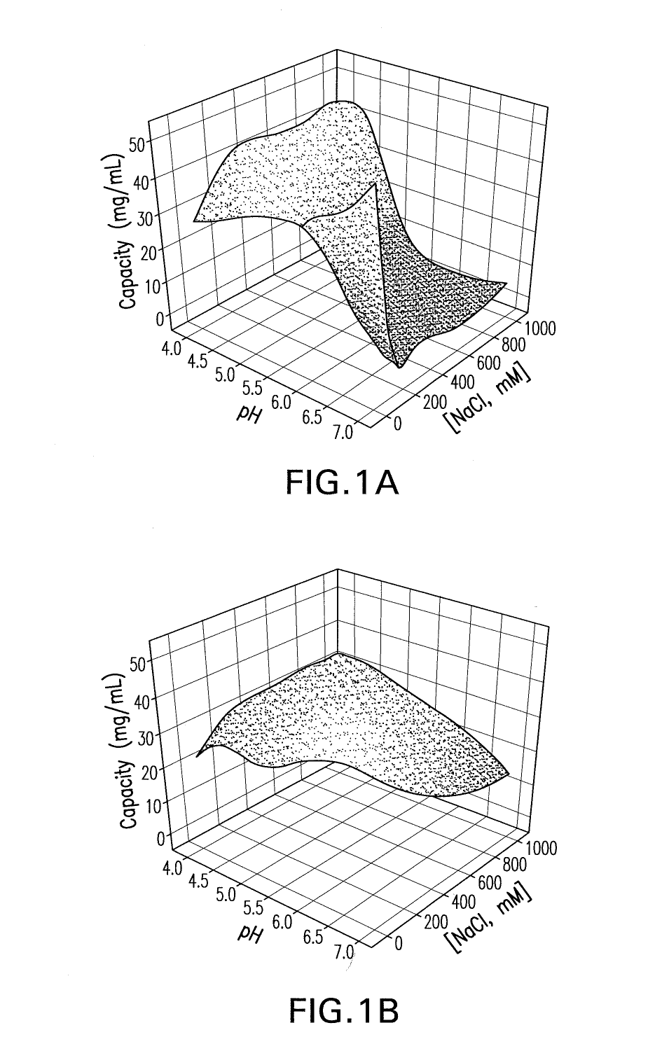

Results can be seen in FIGS. 1A and 1B, in which the binding capacity of the two different purified monoclonal antibodies (anti-ADDL mAb and CS-D7, respectively) on Capto MMC™ is shown as a function of pH and sodium chloride concentration. Although the antibodies used here are both human IgG1κ antibodies with isoelectric points (pI) of 9.0, their binding behavior is significantly different. For the anti-ADDLs mAb, there are regions of sharp contrast in retention, with multiple optima observed at pH 4 and 7. Binding is intolerant to salt concentration at lower pH, but becomes very sensitive to it as the pH increases. In contrast, the binding profile of the CS-D7 mAb is relatively flat, with a single clear optimum at around pH 7 and low ionic strength. These binding differences presumably are due to differences in the hydrophobic properties of the antibodies or to differences in the surface charge distribution (i.e., local patches of charge), since their pIs are similar.

Thus, it is clear that optimization of the mobile phase for mixed mode chromatography needs to occur for each unique monoclonal antibody depending on its surface properties (specifically, its surface charge and hydrophobicity). However, for the CS-D7 and anti-ADDLs mAbs, similar mobile phase conditions can be determined for loading, washing, and elution in which both antibodies can be purified effectively and efficiently, given their similar binding behavior at physiological pH (7.3±1.0). At this pH range, electrostatic interaction between the mAbs and the mixed mode adsorbent appears to predominate when using a sodium chloride salt modifier, while many of the host-cell contaminants do no bind. For other antibodies that are more acidic (lower pI) or that are more or less hydrophobic, the mobile phase condition could be modified, such as loading at a lower pH or changing the salt type, to promote electrostatic and/or hydrophobic binding mechanisms.

### Example 2

### Pre-Screen

Pre-screening experiments were carried out with the purified mAb CS-D7 to establish the approximate ranges of loading pH and salt concentration, ensure product solubility under these conditions, determine the approximate adsorbent capacity, and identify preliminary conditions for product elution. An appropriate loading time must also be established in this stage to ensure that a semi-equilibrium state (≧80% binding) is reached for the representative assessment of column binding conditions. The total load time is controlled by the pipetting flow rate, the volume pipetted, and the number of aspiration-dispense cycles. In this study, a conservative load time of 40 minutes was used to ensure semi-equilibrium binding, while still allowing adequate experimental throughput.

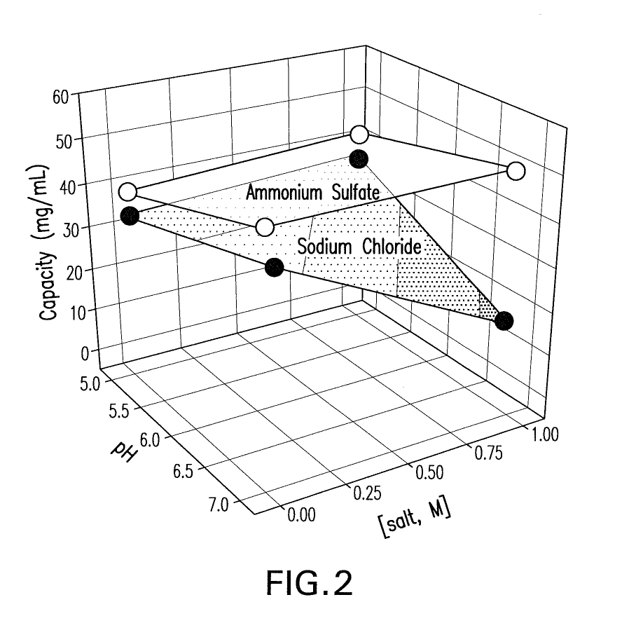

A factorial design was carried out with sodium chloride and ammonium sulfate out to establish the appropriate parameter ranges for pH and salt concentration and to estimate the maximum binding capacity. These results are shown in FIG. 2 with sodium chloride and ammonium sulfate. These salts were considered to be representative of the other two salts, ammonium chloride and sodium sulfate. From these results, the parameter range was extended for the subsequent primary screen to between 4 and 8 for pH and between 0 and 2 M for the salt concentration. However, solubility screens show that the mAb CS-D7 begins to precipitate at higher ammonium sulfate concentrations (Table 2). Therefore, the upper salt concentration was restricted to 1.25 M to ensure mAb solubility. The results from the factorial experiment also indicate that mAb binding decreases with increasing pH and sodium chloride concentration. A column strip solution of 0.5 M NaCl at pH 8.5 was therefore found to be a sufficient for the quantitative elution of the mAb product. This strip buffer was used during the primary screen to examine the reversibility of binding under different load conditions.

TABLE 2
Absorbance at λ = 600 nm in micro-titer
plates for the purpose of screening protein (CS-D7
mAb) solubility at different salt concentrations.

| [Salt], M | 0.00 | 0.50 | 1.00 | 1.25 | 1.50 | 2.00 |
|---|---|---|---|---|---|---|
| NaCl, pH 5 | 0.0213 | 0.0138 | 0.0121 | 0.0111 | — | — |
| NaCl, pH 7 | 0.0185 | 0.0166 | 0.0130 | 0.0129 | — | — |
| (NH4)₂SO₄, | 0.0169 | 0.0113 | 0.0136 | 0.0162 | 0.0207 | 1.2765 |
| pH 5 | | | | | | |
| (NH4)₂SO₄, | 0.0177 | 0.0112 | 0.0129 | 0.0175 | 0.0938 | 1.2936 |
| pH 7 | | | | | | |

(Concentration of mAb after dilution into salt solution is 0.8 mg/mL).

### Example 3

### Primary Screen for Optimization of Solution Conditions

A primary screen was carried out following the pre-screen to evaluate the binding and selectivity of the Capto MMC™ for the mAb CS-D7 and the contaminating host cell proteins. Binding of mAb CS-D7 and host-cell proteins to the Capto MMC™ adsorbent was examined as a function of pH, salt concentration, and salt type in a screening experiment. Thirty-two chromatography experiments (4 runs×8 experiments) were carried out for each salt type, in which each column was overloaded and pH and salt concentration were varied. The pH was varied at five points (4.0, 5.0, 6.0, 7.0, and 8.0) and the salt concentration at six points (0, 0.25, 0.50, 0.75, 1.0, and 1.25). The column feed was exchanged into each solution condition by dilution (≧10-fold) from a concentrated feedstock. In each experiment, the adsorbent was overloaded at a process relevant concentration to provide an estimate of capacity.

Three parameters were examined in this optimization, pH, salt concentration, and salt type. Because of the hydrophobic properties of the MMC ligand, four different salt types were evaluated by varying two cations, NH₄⁺ and Na⁺, with two anions, SO₄²⁻ and Cl⁻. Each pair of ions were selected based on their location in the Hofmeister series (Zhang et al., 2006, Curr Opin Biol Chem 10:658-663), with NH₄⁺ being more lytropic than Na⁺, and SO₄²⁻ being more lyotropic than Cl⁻. The contribution of the buffer (used for pH control) on the chromatography was neglected in these screening studies to keep the number of screening parameters at three. This was done by using an universal buffer mixture of 10 mM sodium acetate, 10 mM sodium phosphate, and 10 mM HEPES, spanning a broad pH range from 4 to 8. Buffer effects can be probed upon defining a narrower pH range of optimal performance.

Samples were diluted a minimum of 10-fold into each solution condition from a concentrated stock solution. Absorbance measurements were made in a 96-halfwell UV transparent plate at 280 nm following each run.

Retention maps of product capacity and impurity binding were generated from the primary screen and used to select the preferred operating mode (flow through or bind-and-elute), the process function (capture, intermediate, or polishing step), and the optimal loading conditions. Product recovery can also be evaluated by carrying out a “strip” elution to ensure that binding is reversible. In choosing lead binding conditions, the importance of each response factor (capacity, purification factor, and recovery) is weighted according to the specified operating mode and process needs. A significant benefit of the retention screen is there is a focus on achieving selectivity on the binding side of the chromatographic separation, as opposed to just on the elution side, which is conventionally the primary focus.

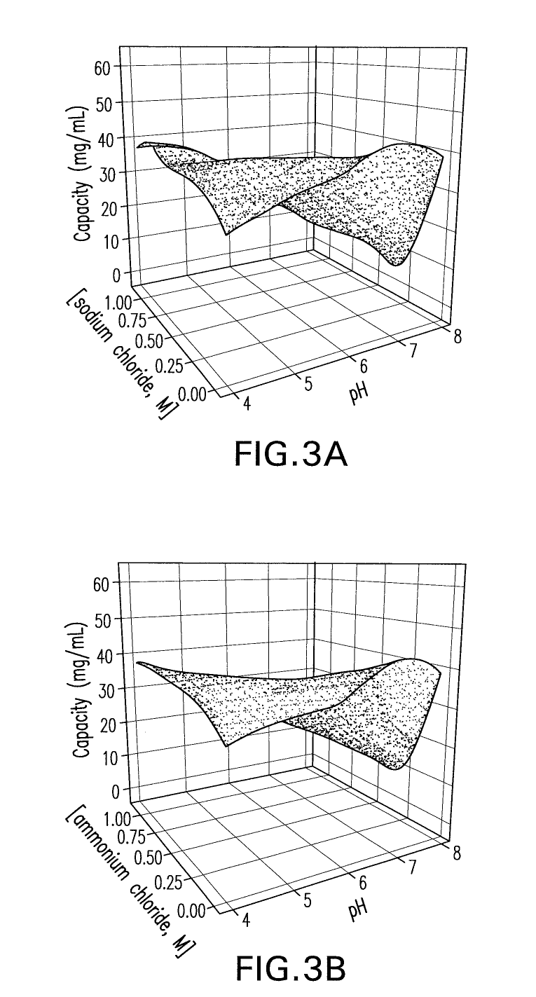

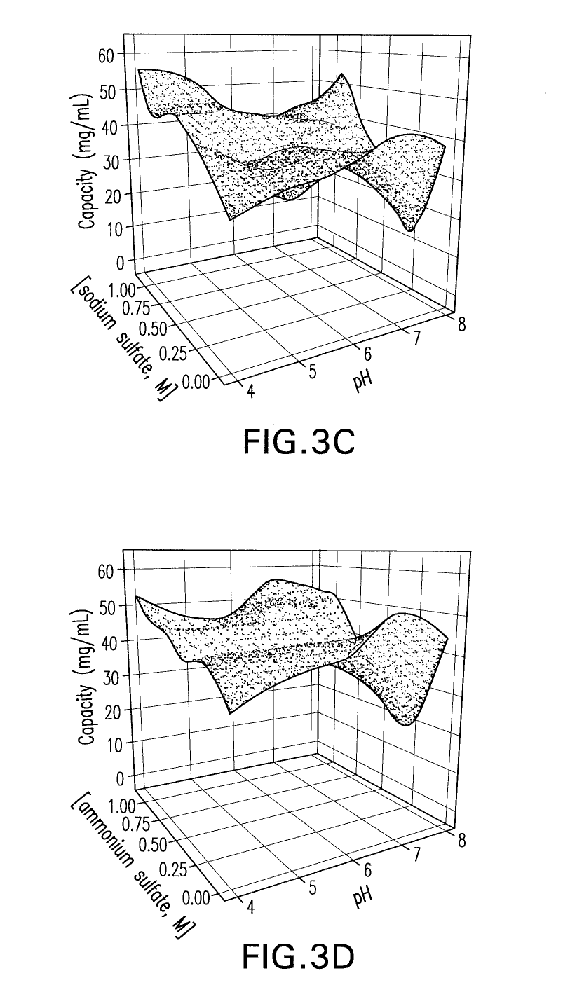

Purified product and impurities were screened separately in this investigation. The 3-D retention map of the mAb CS-D7 in each of the four salts is shown in FIGS. 3A-D. The shape of these retention maps are affected most by the anion, with the map of sodium sulfate resembling ammonium sulfate and sodium chloride resembling ammonium chloride. This is consistent with the observation that the Hofmeister effect is more pronounced with anions. See Zhang et al., 2006, Curr Opin Biol Chem 10:658-663. The small dimples and bumps in the response surfaces are presumed to be from experimental variability. An estimation of the experimental variability was determined from the data points replicated across each experimental set (pH 4, 5, 6, 7, and 8 with no added salt), with the CV<9% (n=4 to 10) in all cases. For the chloride salts, binding is weakened by increasing salt concentration at higher pH (>6), suggesting that electrostatic interactions predominate in this pH range. However, at lower pH (<6), binding is less affected by salt concentration, implying that hydrophobic interactions predominate. In contrast to the chloride salts, increasing the salt concentration for the sulfate ions at higher pH weakens binding only to a certain point (approximately 0.5 M). Then, binding increases again, forming a valley in the response surface. This suggests that hydrophobic interactions are overwhelming electrostatic effects at higher salt concentrations. Binding capacity is the same or increases at lower pH, similar to the chloride ions, with one exception. A second valley is observed at pH 6 and high salt concentration for sodium sulfate, which implies that the sodium and ammonium cations also have some influence on hydrophobic binding. Another difference between the chloride and sulfate salts is that the sulfate salts approach a capacity of 55 mg/mL whereas the chloride salts only approach a capacity of 40 mg/mL.

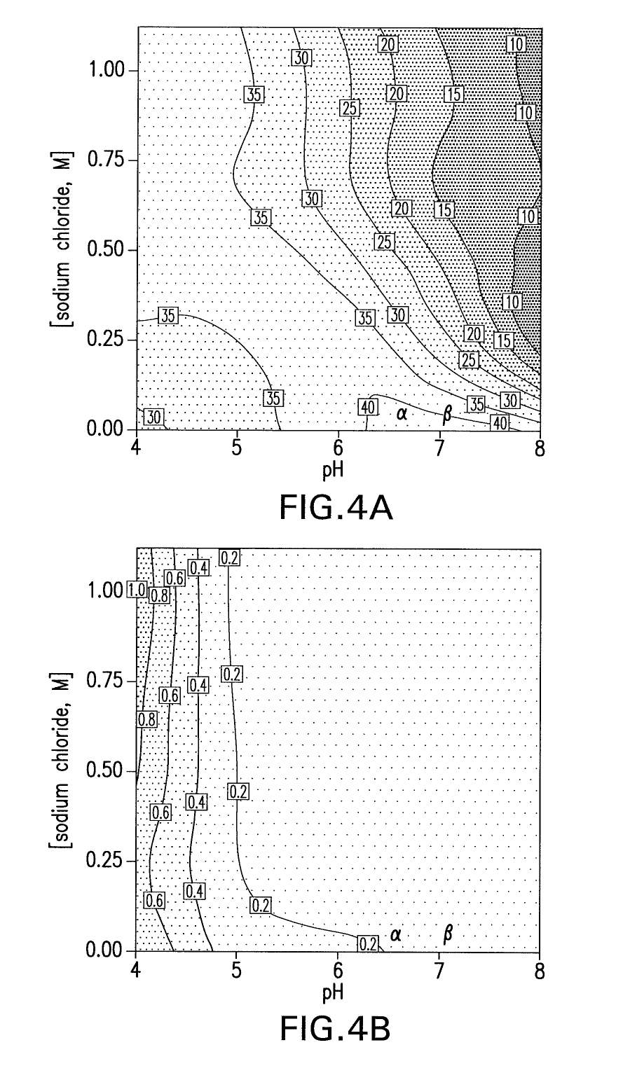

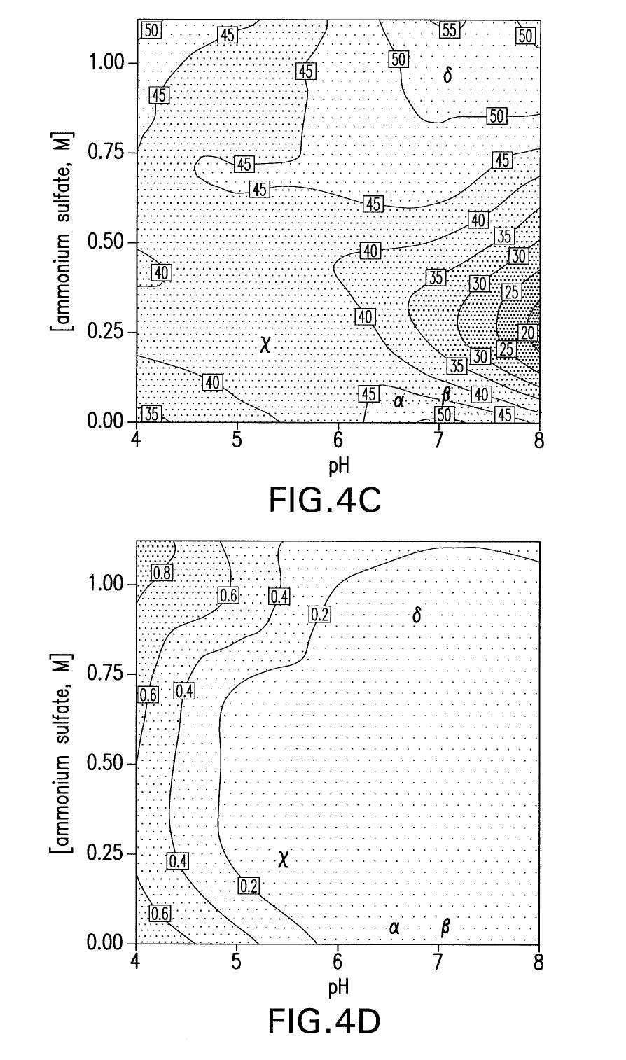

Only the ammonium sulfate and sodium chloride salts were carried forward for the evaluation of host cell protein binding because of the similarity of the anion salt pairs. The binding contour plots of impurity binding are compared with those of the antibody binding in FIGS. 4A-D. Four lead conditions (pH 6.7±0.2 and no added salt, pH 7.1±0.2 and no added salt, pH 5.5±0.2 and 0.25±0.05 M ammonium sulfate, and pH 7.0±0.2 and 0.9±0.05 M ammonium sulfate) were identified. These conditions were selected because they provided sufficiently high mAb capacity (≧40 mg/mL) and low impurity binding (≦0.2 relative host cell protein binding). Of these, the condition yielding the highest capacity was the pH 7.0, 0.9 M ammonium sulfate point. An additional criterion that was considered was the robustness of the operating range around each point (insensitivity to small changes in pH or salt concentration). Specifically, the pH 5.5, 0.25 M ammonium sulfate condition was chosen because of its very robust operating window (wide range of insensitivity of pH and salt concentration). Overall, given the potential for very high clearance of protein impurities, the Capto MMC™ adsorbent appears to be well suited for a capture step in the purification of mAb from Pichia Pastoris cell filtrate, when operated in a bind-and-elute mode.

### Example 4

### Selection of Optimal Conditions Using Statistical Software

A statistical DOE software package can benefit data analysis even if it is not used in the experimental design by enabling multiple variables and/or response factors to be evaluated simultaneously, particularly in cases when this cannot easily be done graphically. In addition, these software packages typically allow specifications to be placed on the data, such limiting the parameter input range, requiring a minimum or maximum value to be met, and weighting responses according to their importance to the purification. In this study, the Design Expert software from Stat-Ease was used to analyze the data from the sodium-chloride and ammonium-sulfate screening experiments. These data, because of their complex response surface, were fit with a cubic polynomial model (the highest order model available in the software). The responses of mAb capacity (maximize this response), impurity binding (minimize this response), and recovery (exceed a minimum threshold) were evaluated in this statistical analysis. The capacity and purification factor responses were weighted with equally high importance, and only recoveries >65% were allowed. No constraints were placed on the parameter input ranges (pH 4 to 8; salt concentration 0 to 125 M). A desirability plot for optimal loading which balances capacity with extent of purification is shown in FIG. 5, with 0 being the least desirable condition and 1 being the most desirable. The favorable loading regions from this statistical evaluation agree well with visual assessment made from the contour plots.

### Example 5

### Secondary Evaluation: Optimization of Lead Conditions

The purpose of the primary retention screen is not only to understand product retention and define the adsorbent capacity but also to attempt to achieve some selectivity on the adsorption side of the chromatographic separation. This is particularly desirable for a capture chromatography in order to increase product capacity (decrease impurity binding) and reduce the burden of separation on the elution step. In the test case examined here, binding conditions were identified for the Capto MMC™ adsorbent that showed very good selectivity for the mAb CS-D7, in which many of the Pichia pastoris host-cell protein impurities were not retained. A secondary evaluation was next performed to optimize these lead conditions, with the primary focus being on optimizing the wash and elution conditions. The methods for the secondary evaluation generally offer greater flexibility than those used in the initial screen, with a larger focus on the wash and elution strategies. A multi-step “staircase” elution method in which the elution strength is incrementally increased in small steps is one way of approximating gradient elution using batch techniques. The retention maps from the primary screen were used to guide the selection of ranges for pH and salt concentration. Adsorbent loading and its impact on the recovery and purification can be explored at this stage.

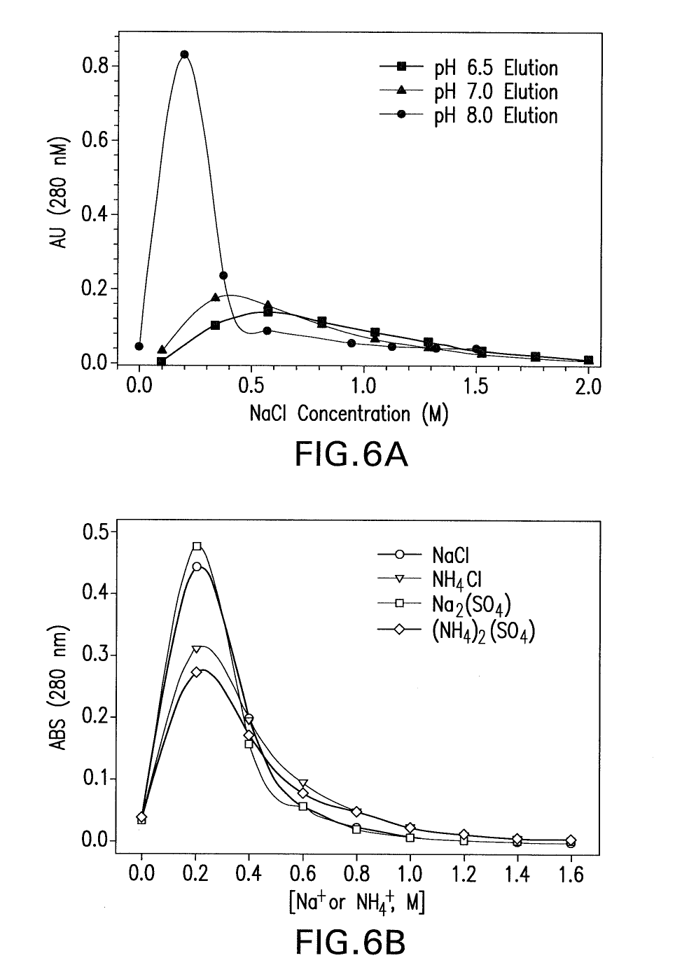

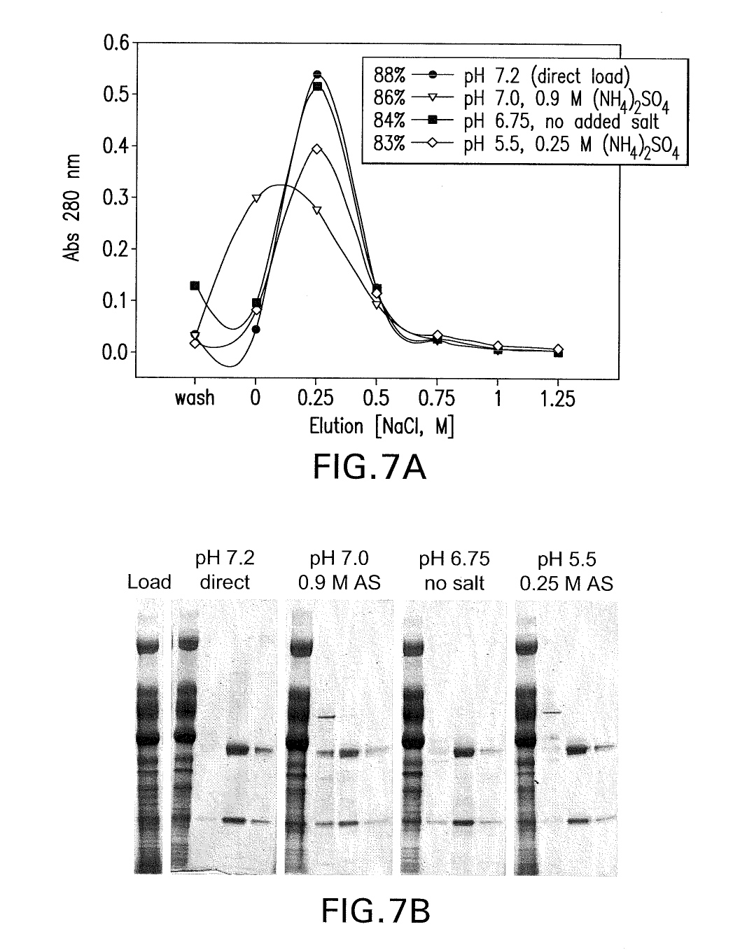

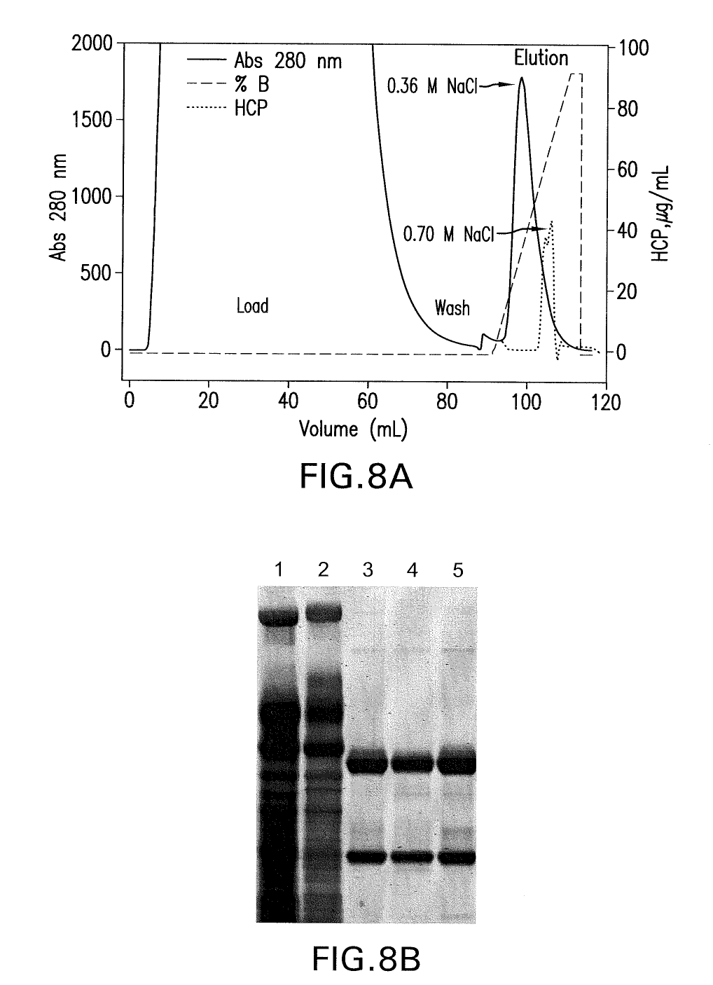

As observed in the retention maps shown in FIGS. 3A-D, mAb adsorption is weakened at higher pH and ionic strength, although binding strength begins to increase again for the sulfate salts at concentrations greater than 0.25 M. FIGS. 6A-B show the effect of pH (6.5-8), salt type, and cation concentration (0 to 1.6 M NH₄+ or Na+) on the elution of the mAb CS-D7. In these experiments, the columns were loaded with cell filtrate at pH 7.1+0.2 and no added salt. Higher pH levels result in a more efficient elution, as does elution with the sodium salts. Sodium chloride is preferred over sodium sulfate however since the mAb CS-D7 is not retained at high salt concentrations. Therefore, it was used for the remainder of the secondary evaluation.

The four optimal loading conditions identified from the primary screen (FIGS. 4A-D and 5) were compared by carrying out a staircase elution (0.25 M/step) with sodium chloride at pH 8. A direct load of the clarified cell filtrate (pH 7.2, conductivity<10 ms/cm) was carried out in lieu of the pH 7.1 (no salt added condition), whereas for the other conditions, the clarified filtrate was exchanged (dialyzed) into the desired solution condition. The elution profile is shown in FIG. 7A along with the % yield as measured by the Octet biosensor assay. Interestingly, for the condition loaded at 0.9 M ammonium sulfate (pH 7.0), the product begins to elute with the decrease in salt concentration (0.9 to 0 M) and the shift in pH (7 to 8), whereas elution for the other three conditions does not begin until the salt concentration is increased to 0.25 M NaCl. This suggests that adsorption in 0.9 M ammonium sulfate is driven primarily by hydrophobic interaction, whereas the interaction in the other three loading conditions is predominantly electrostatic, or a combination of the two. While the recovery is comparable for the four loading conditions (>80%), the extent of purification differs, as observed in FIG. 7B. Several protein contaminants as well as free antibody light chain elute at pH 8 (no salt). As a result, some of the product co-elutes with these impurities for the feed loaded in 0.9 M ammonium sulfate. The purity of each of the products from the other three loading conditions is comparable, with a purity of approximately 90% by SDS-PAGE. Since the direct load is the most convenient of the three conditions, with no buffer exchanged required, it was chosen as the winning condition for verification in a dynamic column chromatography experiment.

### Example 6

### Column Verification of Microscale Conditions

The optimal condition selected from the batch micro-tip chromatographic experiments (direct load of clarified filtrate) was verified in a 1-mL laboratory column operated in a dynamic mode. The crude cell filtrate (pH 7.2±0.2; conductivity<10 mS/cm) was loaded directly onto the column at a residence time of 4 minutes (37.5 cm/h; total load time of 145 minutes) and a loading challenge of 36 mg of mAb per mL of adsorbent. The product was eluted with sodium chloride (pH 8) in a twenty-column-volume linear gradient from 0 to 1 M. The UV chromatogram and gel electrophoresis results are shown in FIGS. 8A-B. Most of the host-cell protein impurities do not bind to the column and are recovered in the column flow-through, as is observed in the microscale purification. The dynamic capacity at 2% breakthrough was determined from this column experiment to be approximately 34 mg/mL adsorbent, in line with the semi-equilibrium capacity of 35-40 mg/mL observed in the micro-tip experiments. The product elutes from the column at about 0.36 M NaCl, also consistent with the micro-tip chromatography (elution between 0.25 and 0.5 M NaCl). For those host-cell proteins that do bind to the column, the majority of them appear to elute either at the start of the gradient or following the mAb elution, at around 0.7 M NaCl. The product purity is about 90% by SDS-PAGE, comparable to a protein-A purified product.

A comparison of the laboratory-scale column purification to the micro-tip purification is shown in Table 3. The product recovery between the two scales is comparable, at around 80%. Although the step yield is below 90%, it is still acceptable, especially given the significant clearance of host cell protein (99.99%) and DNA (97.99%) that is achieved. The host cell protein level in the laboratory column product is about 3-fold lower than that of the micro-tip column, presumably due to the increased resolution gained in carrying out the gradient elution on the column. In the purification of the mAb from the crude filtrate, conditions for operating the chromatography as a capture step were determined that removed greater than 99.99% of the host cell protein and 99.97% of the DNA with a step yield of 82%.

TABLE 3
Summary of results for the purification of mAb from
cell filtrate using Capto MMC ™ chromatography at
micro- and laboratory-scales^{a}.

| | Capto MMC ™ | Capto MMC ™ |
|---|---|---|
| | Micro-Tip Column | Lab-scale Column |
| | (10 μL; Batch) | (1 mL; Dynamic) |
| Step Recovery (%) | 77 | 82 |
| Purity, SDS-PAGE (%) | 88 | 88 |
| Purity, HCP ELISA (ppm) | 5346 | 1624 |
| Host-Cell Protein Clearance (%) | 99.98 | 99.99 |
| DNA clearance (%) | not assayed | 97.99 |

^{a}Adsorbent loading at both scales was approximately 35 mg mAb/mL adsorbent.

What is claimed is:

1. A method of purifying an antibody having an isoelectric point of greater than 7.0 from a mixture containing the antibody comprising the steps of:
(a) applying said mixture onto a solid phase comprising a mixed mode resin containing ligands which comprise a negatively charged part and a hydrophobic part; and
(b) eluting said antibody with an elution buffer containing at least about 0.3 M sodium chloride or about 0.2 to about 0.4 M sodium sulfate, buffered at pH of about 7.8 to about 8.5; and
(c) recovering said antibody from the mixed mode resin.

2. The method of claim 1, wherein said eluting is by
i) step elution with an elution buffer containing ≧about 0.4 M sodium chloride or about 0.2 to about 0.4 M sodium sulfate; or
ii) gradient elution comprising from about 0.3 to about 0.4 M sodium chloride.

3. The method of claim 1, wherein said antibody has an isoelectric point of about 8.5 or greater.

4. The method of claim 1, wherein said antibody has an isoelectric point of about 8.5 to about 10.5.

5. The method of claim 1, wherein said antibody has an isoelectric point of about 8.5 to about 9.5.

6. The method of claim 1, wherein said mixture has not been subject to any preceding chromatographic purification step.

7. The method of claim 1, wherein the buffer is HEPES, MOPS, TRIS, phosphate, BICINE, or triethanolamine.

8. The method of claim 1, wherein prior to step b), the solid phase is washed with a wash buffer selected from the group consisting of Tris, HEPES, MOPS or phosphate in the absence of sodium chloride, at pH 7.0 to 8.0 and a conductivity<5 mS/cm.

9. The method of claim 2, wherein said eluting is performed by step elution with an elution buffer containing about 0.5 to about 1.0 M NaCl buffered at pH 8.0±0.1

10. The method of claim 2, wherein said eluting is performed by gradient elution from 0 to about 1.0 M, 0 to about 0.5 M or about 0.1 to about 0.4 M NaCl buffered at pH 8.0±0.1 over about 5 to about 20 column volumes.

11. The method of claim 1, wherein said eluting is performed by step elution with an elution buffer containing 0.5±0.1 M NaCl at pH 8.0±0.1.

12. The method of claim 1, wherein said negatively charged part is an anionic carboxylate group or anionic sulfo group for cation exchange.

13. The method of claim 1, wherein said solid phase comprising a mixed mode resin is Capto MMC™.

14. The method of claim 1, wherein the mixture is applied directing without adjusting the pH or conductivity.

15. The method of claim 14, wherein the mixture has a pH 7.2±0.3.

16. The method of claim 1, wherein the mixture is applied after adjusting the pH to the pH of the loading solution to a pH of about 6 to about 8 and adjusting the conductivity to <10 mS/cm.

17. The method of claim 16 wherein the pH of the loading solution is adjusted to a pH of about 7.0 to about 7.5.

18. The method of claim 1, wherein ≧90% of the host cell protein and DNA is removed and the yield of monoclonal antibody is ≧70%.

19. The method of claim 18, wherein the yield is ≧80%.

20. The method of claim 1, wherein said fermentation broth is clarified by centrifugation or depth filtration.

21. The method of claim 1, wherein the antibody is purified to a purity of ≧80% as assessed by gel electrophoresis.

22. The method of claim 21, wherein the antibody is purified to a purity of ≧85%.

23. The method of claim 1, further comprising a step of purification or filtration after step b).

24. The method of claim 23, wherein said step of purification or filtration is microfiltration or sterile filtration.

25. The method of claim 1, wherein said antibody is a Staphylococcus aureus CS-D7 target region specific monoclonal antibody.

26. The method of claim 1, wherein said antibody is CS-D7.

27. The method of claim 1, wherein said mixture is a Pichia pastoris fermentation broth.

28. The method of claim 25, wherein said Pichia pastoris fermentation broth is from a glycoengineered strain.

29. The method of claim 28, wherein said glycoengineered strain provides predominantly N-linked glycans of any of the following types: Man₅GlcNAc₂, GlcNAc₂Man₃GlcNAc₂, Gal₍₀₋₂₎GlcNAc₂Man₃GlcNAc₂ and NANA₍₁₋₂₎Gal₂GlcNAc₂Man₃GlcNAc₂.
